(19) United States
(12) Patent Application Publication
(10) Pub. No.: US 2013/0283502 A1
(43) Pub. Date: Oct. 31, 2013

(54) NOVEL REMOVABLE CLOTHING

(71) Applicant: Rochelle DENNING, Springfield, NJ (US)

(72) Inventor: Rochelle DENNING, Springfield, NJ (US)

(21) Appl. No.: 13/930,612

(22) Filed: Jun. 28, 2013

Related U.S. Application Data
(63) Continuation-in-part of application No. 13/827,283, filed on Mar. 14, 2013.
(60) Provisional application No. 61/611,887, filed on Mar. 16, 2012.

(51) Int. Cl. A41B 9/00; A61F 13/72; A41B 9/12

(52) U.S. Cl. CPC A41B 9/001; A41B 9/12; A61F 13/72
USPC 2/403; 604/396

(57) ABSTRACT

The invention generally relates to easily removable clothing that is intended for individuals with limited mobility. The invention further relates to easily and safely removable clothing for individuals that may be recovering from surgery or suffer from a disability which limits their physical mobility. The invention also relates to pull-tab systems that may be combined with clothing or accessories in order to facilitate easier and simpler removal and wear.

### CROSS REFERENCE TO RELATED APPLICATIONS

This application is a continuation-in-part of U.S. application Ser. No. 13/827,283, filed on Mar. 14, 2013, which claims the benefit of U.S. Provisional Application No. 61/611,887, filed on Mar. 16, 2012, the contents of each of which are herein incorporated by reference in their entireties.

### FIELD OF THE INVENTION

The field of the invention generally relates to easily removable clothing that is intended for individuals with limited mobility. The field of the invention further relates to easily and safely removable clothing for individuals that may be recovering from surgery or suffer from a disability which limits their physical mobility.

### BACKGROUND OF THE INVENTION

Often, individuals with limited mobility require assistance with putting on, and taking off, clothing. The issue of movement can be exacerbated when afflicted individuals live alone with no one to aid them in daily morning dressing or rearrangement of their clothing after a trip to the bathroom or shower. For example, an individual may have difficulties using their lower extremities in order to get undergarments or outer garments up the leg openings and over or under the buttock area into a comfortable proper position. The inability to accomplish this seemingly simple, but very necessary task may be the result of temporary, or permanent, loss of flexibility to the lower extremities.

A wide variety of devices have been developed to assist physically challenged individuals in their daily lives. Some devices are used to retrieve items from the floor, tables, or shelves, and have been modified to assist an individual in putting on socks and shoes. Certain dressing aids have been developed for a variety of specific uses: long-handled shoe horns to help those that cannot easily bend from the waist or plastic sleeves to assist in putting on socks and stockings. Such devices may also include a “pull-stick”to move the sock/stocking into position once it is positioned over the foot and ankle. Dressing sticks comprising a stiff shaft and soft “hook” at the opposite end of the grip area are used by some to help pull-up pants legs.

The marketplace has largely ignored the needs of mobility-impaired individuals. For example, many popular undergarments are primarily intended for individuals that are incontinent. The result has been clothing that differs significantly from traditional clothing in choice of style and fabrics, for example. In addition, these types of articles of clothing lack a design that can assist an individual with dressing him or herself without the aid of a third party. Accordingly, there is a strong market need for clothing with functional ease, but which is combined with the style and fabrics normally associated with traditional clothing, e.g., undergarments.

### BRIEF DESCRIPTION OF THE DRAWINGS

FIG. 1 is a front view of the women's underwear that is one embodiment of the invention.

FIG. 2 is an above view, of the interior of the women's underwear.

FIG. 3 is an above view of the exterior of the women's underwear.

FIG. 4 is a side view of the women's underwear wherein the connective side strips are fastened and engaged.

FIG. 5 is a side view of the women's underwear wherein the connective side strips are unfastened and disengaged.

FIG. 6 is a front view of the men's briefs that are one embodiment of the invention.

FIG. 7 is an above view, of the interior of the men's briefs.

FIG. 8 is an above view of the exterior of the men's briefs.

FIG. 9 is a side view of the men's briefs wherein the connective side strips are fastened and engaged.

FIG. 10 is a side view of the men's briefs wherein the connective side strips are unfastened and not engaged.

FIG. 11 is front view of the men's boxer briefs that are one embodiment of the present invention.

FIG. 12 is an above view, of the interior of the men's boxer briefs.

FIG. 13 is an above view of the exterior of the men's boxer briefs.

FIG. 14 is a side view of the men's boxer briefs wherein the connective side strips are fastened and engaged.

FIG. 15 is a side view of the men's boxer briefs wherein the connective side strips are unfastened and not engaged.

FIG. 16 is front view of the men's boxer underwear that is one embodiment of the present invention.

FIG. 17 is an above view, of the interior of the men's boxer underwear.

FIG. 18 is an above view of the exterior of the men's boxer underwear.

FIG. 19 is a side view of the men's boxer underwear wherein the connective side strips are fastened and engaged.

FIG. 20 is a side view of the men's boxer underwear wherein the connective side strips are unfastened and not engaged.

FIG. 21 is an exploded or close detailed view of a portion of the pull-tab system that is one embodiment of the present invention.

### SUMMARY OF THE INVENTION

It is an advantage of the present invention that a user has the freedom to don and remove clothing without the assistance of a caregiver or some other type of assistant. As a result the user is provided increased dignity and independence.

A further advantage of the present invention is that individuals with limited mobility will have little difficulty wearing and removing the articles of clothing, e.g., garments and undergarments, disclosed herein.

Still yet another advantage of the present invention is that individuals with limited mobility require very little motion or movements to put on or remove the garments and undergarments disclosed herein. The amount of motion, bending, etc., required to remove or put the garments or undergarments on is significantly less for a user than traditional articles of clothing. For example, a user may put on or remove the undergarments or garments disclosed herein while laying supine. Moreover, because the undergarments or garments disclosed herein may attach, in certain embodiments, on the side of the user, the user can put on or take off the articles of clothing while laying supine and without having to arch their back. This is one example of the “significantly less” motion that is required to wear or remove the articles of clothing disclosed herein compared to traditional garments or undergarments.

An embodiment of the invention comprises garments which utilize one or more pull tabs and one or more connective strips (e.g., connective side strips) in order to secure the garment, wherein the connective strips further comprise connective means (e.g., hook and loop apparatus) and are easily accessible for the user, and the user can don or remove the garment with ease and limited movement.

Another embodiment of the invention comprises undergarments that utilize one or more pull-tab connective strips (e.g., connective side strips) in order to secure the undergarment to body of the user. The connective strips further comprise connective means (e.g., hook and loop apparatus) and are typically located on the sides of the garment.

Another embodiment of the invention comprises a pull-tab system, wherein the pull tab system comprises one or more connective strips which further comprises corresponding connective means (e.g., hook and loop apparatus) and wherein a pull tab is fastened (e.g., stitched) medially to the top of the outermost connective strip allowing a user to pull down on said in order to remove the garment with out having to bend or move in any significant way. In one embodiment a user is able to insert a thumb or finger through the pull tab to join or disengage the connective strips. In one embodiment a user is able to insert a finger or thumb into the pull tab loop to pull down in order to easily and quickly disengage the connective strips. The pull-tab system may be combined with most articles of clothing, (e.g., garments or undergarments)

Yet another advantage of the invention is that the connective strips disclosed herein may be positioned in such a way where they can be close in proximity to the user or wearer but that the sometimes abrasive connective means do not grate against the skin of the user.

### DETAILED DESCRIPTION

The present invention provides articles of clothing, e.g., garments or undergarments, which can be made from natural or synthetic materials and are easy to wear and remove. It is an advantage that the undergarments described herein may in some embodiments be made from traditional fabrics and materials typically associated with the manufacture of typical articles of clothing, e.g., garments or undergarments.

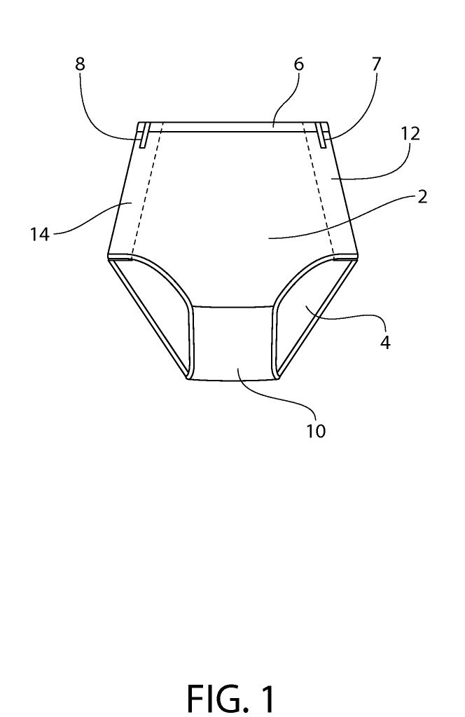

In one embodiment the invention is directed to toward Removable Undergarment-1, e.g., any of FIG. 1-FIG. 21, wherein the undergarment comprises: a natural or synthetic material or fabric, a waistband, one or more connective strips (e.g., connective strips located at a user's left and right sides), wherein the connective strips further comprise connective means (e.g., hook and loop apparatus, e.g., VELCRO®) and a pull tab (e.g., wherein the pull tab is attached to the outermost portion of the connective strip). In one embodiment, at least one side connective strip (located on the user's left or right side) runs the entire length of the side of the undergarment. In one embodiment the length of the connective strips may be designed in order to adjust for the amount of desired connective strength. In another embodiment, adjusting the connective means will also allow for one to design an undergarment of varying strength (e.g., depending upon the strength of the connective means the undergarment will be easier or more difficult to remove).

For example Removable Undergarment-1 also includes:
1.1 Undergarment 1 wherein the undergarment is cloth, fabric and/or any woven material
1.2 Any of the aforementioned undergarments wherein the undergarment is composed of any known synthetic material or fiber.
1.3 Any of the aforementioned undergarments wherein the undergarment is rayon, acetate, nylon, acrylic, polyester, olefin, spandex, lastex.
1.4 Any of the aforementioned undergarments wherein the undergarment is a natural material or a blend of a natural and synthetic material.
1.5 Any of the aforementioned undergarments wherein the undergarment is cloth, fabric, and/or some woven or non-woven material.
1.6 Any of the aforementioned undergarments wherein the undergarment is made of cotton, silk, wool, and/or a blend of any of other fibers listed herein.
1.7 Any of the aforementioned undergarments wherein the pull tabs of the are composed of any material known in the art, e.g., an elastic or elastic polymer.
1.8 Any of the aforementioned undergarments wherein the pull tab is stiched to the top, medial (e.g., center or middle) portion, of a first connective strip.
1.9 Any of the aforementioned undergarments wherein connective means is selected from the group consisting of: snap fasteners, hook-and-loop fasteners, magnets, buttons, zippers, VELCRO®, lacings, buckles, toggle fasteners, clips, adhesive tape, and clasps.
1.10 Any of the aforementioned undergarments wherein the undergarment is selected from the group consisting of: long underwear, sleeveless shirts, men's briefs, men's boxer shorts, men's boxer briefs, women's underwear (e.g., women's briefs), brassieres, girdles, and nightgowns.
1.11 Any of the aforementioned undergarments wherein at least one pull tab is located in the medial portion (e.g., middle or center portion, and toward one end, e.g., the top,) on at least one connective strip (e.g, first left or first connective right strips), and wherein a user may insert a thumb or finger through the pull tab, and wherein an applied force on the pull tab (e.g., upward, downward, or outward force) is sufficient to engage or disengage the connective strip from, or to engage the connective strip to, a second corresponding or complementary connective strip.
1.12 Any of the aforementioned undergarments wherein the applied force sufficient to engage or disengage the connective strip is about 10-15 lbs/sq inch, and wherein the pull tab is able to spread the distribution of the force (e.g., evenly distribute) felt from the connective resistance of the engaged connective strips the thumb or finger of a user which then lessens the perceived degree of force which a user believes is necessary to disengage the strips from one another.
1.13 Any of the aforementioned undergarments wherein the pull tab is attached to outermost portion of a first connective strip, and wherein the pull tab is located in a position that creates the greatest degree of leverage (e.g., a position which is toward one end or the other of the connective strip, e.g., the top of the connective strip) for pulling the connective strips apart and wherein the pull tab is located in the medial portion, e.g., center or middle, of the connective strip on the outermost portion.
1.14 Any of the aforementioned undergarments wherein the undergarment remains in one piece (e.g., not two disjointed pieces) even when the connective strips are disengaged.
1.15 Any of the aforementioned undergarments wherein the undergarment comprises: left and first connective right side strips, left and second connective right side strips, wherein at least one pull tab is attached to the outermost portion of at least one of the left or first connective right side strips.
1.16 Any of the aforementioned undergarments wherein the undergarment is any of FIG. 1-FIG. 20.
1.17 Any of the aforementioned undergarments, wherein the undergarment is an easily removable undergarment that is easily removed or worn by an individual with limited physical mobility.
1.18 Any of the aforementioned undergarments, wherein the undergarment comprises a pad (e.g., an absorbent pad).
1.19 The undergarment of 1.18 wherein the pad is optional.
1.20 The undergarment of 1.18 or 1.19, wherein the pad is an incontinence pad, wherein the pad can be either a male or female incontinence pad.
1.21 The undergarment of 1.18-1.20, wherein the pad is a separate replaceable insert, and wherein the pad is disposable.
1.22 The undergarment of 1.18-1.20, wherein the pad is permanently attached or coupled with the undergarment, and wherein the pad is washable and reusable (e.g., an absorbent liner).
1.23 The undergarment of any of 1.18-1.22 wherein the pad comprises the following components: an outerwrap, topsheet, acquisition layer, core, absorbent layer, backsheet, elastic, release tape, and adhesives.
1.24 The undergarment of any of 1.18-1.23, where the pad comprises the following materials: polypropylene, and non-woven polypropylene.
1.25 The undergarment of 1.24, wherein the pad further comprises polyester, cellulose, and spandex.
1.26 The undergarment of any of 1.18-2.25, wherein the incontinence pad is a shaped incontinence pad.

In one embodiment the invention is directed to toward Removable Garment-2, wherein Garment-2 (e.g., shirt, pants, socks) comprises a natural or synthetic material or fabric, and one or more connective strips (e.g., side connective strips), wherein the connective strips further comprise connective means (e.g., hook and loop apparatus, e.g., VELCRO®) and a pull tab. In one embodiment the length of the connective strips may be designed in order to adjust for the amount of desired connective strength. In another embodiment, adjusting or substituting the connective means will also allow for one to design a garment of varying strength (e.g., depending upon the strength of the connective means the undergarment will be easier or more difficult to remove). Depending on the garment, the connective strips may be located anywhere on the garment (e.g., shirt, pants, socks) that would maximize the ease of a user to don and remove the garment.

For example Removable Garment-2 also includes:
2.1 Garment-2 wherein the garment is cloth, fabric and/or any woven material
2.2 Any of the aforementioned garments wherein the garment is composed of any known synthetic material or fiber.
2.3 Any of the aforementioned garments wherein the garment is rayon, acetate, nylon, acrylic, polyester, olefin, spandex, lastex.
2.4 Any of the aforementioned garments wherein the garment is a natural material or a blend of a natural and synthetic material.
2.5 Any of the aforementioned garments wherein the garment is cloth, fabric, and/or some woven or non-woven material.
2.6 Any of the aforementioned garments wherein the garment is made of cotton, silk, wool, and/or a blend (e.g., cotton fiber blend) of any of other fibers listed herein.
2.7 Any of the aforementioned garments wherein the pull tabs of the are composed of any material known in the art, e.g., an elastic or elastic polymer.
2.8 Any of the aforementioned garments wherein the pull tab is stitched to the top, medial (e.g., center or middle) portion, of at least one connective strip (e.g., first connective side strip).
2.9 Any of the aforementioned undergarments wherein connective means is selected from the group consisting of: snap fasteners, hook and-loop fasteners, magnets, buttons, zippers, VELCRO®, lacings, buckles, toggle fasteners, clips, adhesive tape, and clasps.
2.10 Any of the aforementioned garments wherein the garment optionally comprises a waistband.
2.11 Any of the aforementioned garments wherein the garment is selected from the group consisting of: boots, coats, dresses, gloves, hats, jackets, jeans, pajamas, pants, raincoat, scarves, shirts, shoes, skirt, slacks, slippers, socks, stockings, suits, sweaters, sweatshirts, swimsuits, t-shirt, tie, trousers.
2.12 Any of the aforementioned garments wherein at least one pull tab is located medially (e.g., medially and toward the top of the connective strip) on at least one connective strip, and wherein a user may insert a thumb or finger through the pull tab, and wherein an applied force on the pull tab (e.g., upward or downward force) is sufficient to engage or disengage the connective strip from, or to, a second corresponding connective strip.
2.13 Any of the aforementioned garments wherein the applied force sufficient to engage or disengage the connective strip is about 10-15 lbs/sq inch, and wherein the pull tab is able to spread the distribution of the force (e.g., evenly distribute) felt from the connective resistance of the engaged connective strips on the thumb or finger of a user which then lessens the perceived degree of force which a user believes is necessary to disengage the strips
2.14 Any of the aforementioned garments wherein the pull tab is attached to outermost portion of a first connective strip, and wherein the pull tab is located in a position that creates the greatest degree of leverage (e.g., a position which is toward one end or the other of the connective strip, e.g., the top of the connective strip) and wherein the pull tab is located in the medial portion of the connective strip on the outermost portion.
2.15 Any of the aforementioned garments wherein the garment remains in one piece single piece even when the connective strips are not engaged.
2.16 Any of the aforementioned garments, wherein the garment is an easily removable garment that is easily removed or worn by an individual with limited physical mobility.
2.17 Any of the aforementioned undergarments, wherein the undergarment comprises a pad (e.g., an absorbent pad).
2.18 The undergarment of 2.17 wherein the pad is optional.
2.19 The undergarment of 2.17 or 2.18, wherein the pad is an incontinence pad, and wherein the pad can be either a male or female incontinence pad.
2.20 The undergarment of 2.17-2.19, wherein the pad is a separate replaceable insert, and wherein the pad is disposable.
2.21 The undergarment of 2.17-2.19, wherein the pad is permanently attached or coupled with the undergarment, and wherein the pad is washable and reusable (e.g., an absorbent liner).
2.22 The undergarment of any of 2.17-2.21 wherein the pad comprises the following components: an outerwrap, topsheet, acquisition layer, core, absorbent layer, backsheet, elastic, release tape, and adhesives.
2.23 The undergarment of any of 2.17-2.22, where the pad comprises the following materials: polypropylene, and non-woven polypropylene.
2.24 The undergarment of 2.23, wherein the pad further comprises polyester, cellulose, and spandex.
2.25 The undergarment of any of 2.17-2.24, wherein the incontinence pad is a shaped incontinence pad.

In one embodiment the invention comprises Pull-Tab System-3, wherein System-3 comprises one or more pull-tabs and one or more connective strips, and wherein the connective strips further comprise connective means (e.g., complementary connective means, e.g., hook and loop apparatus), wherein the Tab System may combined with an article of clothing or an accessory in order to design an article of clothing or accessory to be easier to remove for a person with limited mobility. In one embodiment the pull-tab is attached (e.g., stitched) to the outermost connective strip (e.g., the connective strip which does not directly contact the user or wearer). In another embodiment the pull-tab is stitched to the medial portion (e.g., center or middle) toward one end (e.g, toward the top) of at least one connective strip (e.g., the outermost connective strip, e.g., a first connective strip).

For example Pull-Tab System-3 also includes:
3.1 System-3 wherein the system is combined with an article of clothing (e.g, garment or undergarment).
3.2 Any of the aforementioned systems, wherein the article of clothing is selected from the group consisting of: boots, coats, dresses, gloves, hats, jackets, jeans, pajamas, pants, raincoat, scarves, shirts, shoes, skirt, slacks, slippers, socks, stockings, suits, sweaters, sweatshirts, swimsuits, t-shirt, tie, trousers, long underwear, sleeveless shirts, men's briefs, men's boxer shorts, men's boxer briefs, women's underwear (e.g., women's briefs), brassieres, girdles, and nightgowns.
3.3 Any of the aforementioned systems wherein the connective means are selected from the group consisting of: snap fasteners, hook-and-loop fasteners, magnets, buttons, zippers, VELCRO®, lacings, buckles, toggle fasteners, clips, adhesive tape, and clasps.
3.4 Any of the aforementioned systems wherein the pull-tab allows a user to don and remove clothing without bending at the waist.
3.5 Any of the aforementioned systems wherein the pull tab is positioned at the top of the connective strip, is positioned medially, and allows a user to simply pull-down in order to disengage the attached connective means (e.g., corresponding hook and loop apparatus)
3.6 Any of the aforementioned systems wherein the connective means (e.g., hook and loop apparatus) does rub against the skin of the user.
3.7 Any of the aforementioned systems wherein at least one pull tab is located medially (e.g., medially and toward the top of the connective strip) on at least one connective strip, and wherein a user may insert a thumb or finger through the pull tab, and wherein an applied force on the pull tab (e.g., upward or downward force) is sufficient to engage or disengage the connective strip from, or to, a second corresponding connective strip.
3.8 Any of the aforementioned systems wherein the applied force sufficient to engage or disengage the connective strip is about 10-15 lbs/sq inch, and wherein the pull tab is able to spread the distribution of the force (e.g., evenly distribute) felt from the connective resistance of the engaged connective strips the thumb or finger of a user which then lessens the perceived degree of force which a user believes is necessary to disengage the strips
3.9 Any of the aforementioned systems wherein the pull tab is attached to outermost portion of a first connective strip, and wherein the pull tab is located in a position that creates the greatest degree of leverage (e.g., a position which is toward one end or the other of the connective strip, e.g., the top of the connective strip) and wherein the pull tab is located in the medial portion of the connective strip on the outermost portion.
3.10 Any of the aforementioned systems wherein one of skill in the art redesigns an existing piece of article of clothing or accessory in order make it easier to remove for someone with limited mobility.
3.11 Any of the aforementioned systems wherein the system is used with any of FIG. 1-FIG. 20.
3.12 Any of the aforementioned systems wherein the system is used or combined with any of Undergarment-1, 1.1-1.25, or Garment-2, 2.1-2.24.

As used herein, “securing means” or “connective means” or “connection means” refers to a fastener or connective device (e.g, which are attached to a connective strip). It is contemplated by the present invention that any of the embodiments described herein may comprise connective means which include: snap fasteners, hook-and-loop fasteners, magnets, buttons, zippers, VELCRO®, lacings, buckles, toggle fasteners, clips, adhesive tape, and clasps. The figures and drawings presented herein depict connective means that are hook and loop. However, the present invention contemplates the use of any connective means that is generally known in the art.

As used herein, the terms “tab system” and “pull tab system”, may be used interchangeably. In some instances, “pull tab system” or “tab system” refers to a system which comprises a pull-tab (e.g., a loop) attached to the outside of a connective side strip. “Pull tab system” or “tab system” may also refer to a system which comprises a pull tab and one or more pairs of connective strips (e.g., 2, 4, 6, 8, 10, 12), and wherein each strip further comprises connective means (e.g., hook and loop apparatus) which are intended to attach to a complementary opposing connective means found on a complementary connective strip.

As used herein, it is understood that in some portions of the disclosure that terms “garment” and “undergarment” are both considered articles of clothing, and, as such, the terms may be used interchangeably. In some portions, the terms “garment” and “undergarment” are discussed separately simply for purposes of clarity, simplicity, and/or ease of understanding.

As used herein, the terms “top” and “bottom” will be readily apparent to a user or one of skill in the art. For example, in the context of undergarments, the term “top” refers to an end portion of the connective strip which is close, or in close proximity, to the waist or hip area of the user. Additionally, for example, again in the context of undergarments, the term “bottom” will refer to an end portion of the connective strip which is close, or in close proximity to, the user's thigh or knee. In the case of garments (e.g., shirts, pants, etc.) the terms “top” and “bottom” will likely apply in the same manner but may change due to the type of garment being worn. In some cases “top” may refer to the portion of the connective strip where the attachment of the pull tab creates the greatest amount of leverage for removal.

As used herein, the term “connective side strip” or “connective strip” refers to the portion of the garment, undergarment, or pull tab system, which comprises connective means and is responsible for actually fastening the article of clothing. In some embodiments, portions of the “waistband” and “connective strip” may overlap. For example, in some embodiments a pull-tab may be attached to a connective strip but is also directly attached to a portion of a waistband that overlaps with the connective strip (e.g., the top of the connective strip). In such a case, the pull-tab is attached to the connective strip for purposes of description herein.

As used herein, the terms “connective strip” or “connective panel” or “connection panel” are used interchangeably, and simply refer to the portion or piece of the garment or undergarment or pull tab system which comprises the connective means. For example, the terms “connective strip”, “connective panel” or “connection panel” can be used interchangeably for any of the embodiments disclosed herein e.g., Undergarment-1, 1.1-1.26; Garment-2, 2.1-2.25, or Pull Tab System-3, 3.1-3.12, or any of FIG. 1-FIG. 21.

In further embodiments, the “connective strip”, “connective panel” or “connection panel” is a vertical or longitudinal band or region, wherein the top of the band or region is situated near or about the user's hip/waist area and the bottom of the band or region is situated near or about the user's thigh. In some embodiments the bottom is situated near or about the user's knee. In these embodiments it is contemplated that a pull tab is associated or coupled to the outer surface (e.g., the outermost portion of the connective strip) of the “connective strip”, “connective panel” or “connection panel”. It is contemplated that these embodiments may be used with any of the embodiments disclosed herein, e.g., Undergarment-1, 1.1-1.26; Garment-2, 2.1-2.25, or Pull Tab System-3, 3.1-3.12, or any of FIG. 1-FIG. 21.

In a further embodiment it is contemplated that the “connective strip”, “connective panel” or “connection panel” is a vertical or longitudinal band or region, wherein “vertical band”, “vertical region”, “longitudinal band” or “longitudinal region” are “substantially rectangular”. As used herein, the term “substantially rectangular” is intended to mean having two approximately parallel sides, and width shorter than length. The side joining the two parallel sides can be any convenient angle relative to the two approximately parallel sides and relative to the undergarment, garment, or pull tab system which is used.

It is contemplated that the connective strips disclosed herein may be “substantially rectangular” connective strips. For example, any of the embodiments disclosed herein, e.g., any of Undergarment-1, 1.1-1.26; Garment-2, 2.1-2.25, or Pull Tab System-3, 3.1-3.12, or any of FIG. 1-FIG. 21, may have “substantially rectangular connective strips”. It is an advantage of the substantially rectangular connective strips that they provide secure fastening and that their location allows for an individual to easily access them in order to remove the garment.

As used herein, the term “panel” may refer to a piece of fabric or material (e.g., piece of cloth) that is used to form the basic structure or body of an undergarment or garment.

As used herein, “persons with limited mobility” or “individuals with limited mobility” or “individual with limited mobility” are used interchangeably. These terms may refer to, but are not limited to, e.g, individuals with a physical disability from injury, individuals that suffer from a physical disability resulting from a disease or illness, older individuals that may have limited mobility due to advanced age, individuals recovering from surgery. Other applicable individuals for which these terms pertain would be readily understood by one of skill in the art.

As used herein, the terms “pads” or “absorbent pads” or “incontinence pads” and the like may be used interchangeably. The terms describe pads that are used to absorb urine or other liquids or bodily fluids. The terms “pads” and the like can include panty-liners and inserts. It is contemplated that in some aspects the pads can be reusable and laundered to use many times. In some aspects the pads are disposable. It is contemplated that in some aspects the undergarment or garment, e.g., any of Undergarment-1, 1.1-1.26; Garment-2, 2.1-2.25, or Pull Tab System-3, 3.1-3.12, or any of FIG. 1-FIG. 21, include, but are not limited to, a pad (e.g., an absorbent pad, e.g., an incontinence pad).

FIGS. 1-21 are intended to be depictions of various embodiments of the present invention and are not meant to be limiting in any way.

FIGS. 1-5 depict one embodiment of the present invention wherein the undergarment is woman's underwear. In this embodiment the underwear can be made of woven, nonwoven or knitted fabric or any fabric or material that is normally used in traditional undergarments. However, the vertical connective side strips allow for a secure connective which can easily be put on and then removed.

FIG. 1 is front view of the women's underwear in this embodiment. The underwear has a similar fit to traditional undergarments that are normally secured to an individual by an elastic band, and contains a front strip 2 which is attached to a crotch strip 10, which is in turn attached to a back strip 4. The figure depicts a woman's undergarment that is secured to an individual in part via a waistband 6 in addition to a first connective left side strip 12 and a first connective right 14 side strip. The figure details the left pull tab 7, and the right pull tab 8, of the undergarment. The pull tabs, 7-8, allow for an individual to easily remove the undergarment. It is one advantage of the present invention that an individual may pull the tabs without bending at the waist. The front 2, rear 4, and crotch strips 10 are connected by stitching or other means known in the art. The dashed line simply represents the boundary the connective side strips.

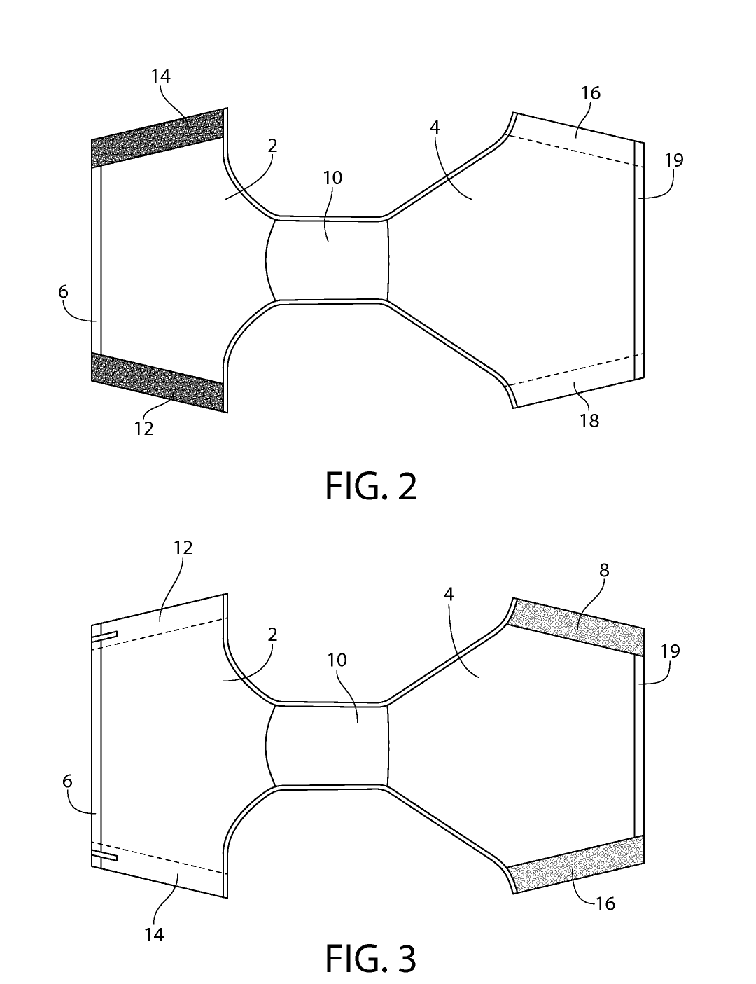

FIG. 2 is an above view, of the interior of the women's underwear. FIG. 3 is an above view of the exterior of the women's underwear. FIGS. 2 and 3 both illustrate the women's underwear laid flat. The connective portions of the garment are located on the sides and allow the user to connect the garment together at around the side of the hip area. In this embodiment FIG. 2 shows that the first connective left side strip 12 and first connective right side strip 14 each comprising connective means which are loops (the loops are designated by black shading). The second connective left side strip 18, and second connective right side strip 16 each comprise the complementary hooks (not shown). The dashed line of FIG. 2 simply indicates the boundary of the second connective side strips. FIG. 3 details that the waistband (front portion 6, back portion 19) is located at the waist portion of the garment and fits around the user's waist. FIG. 3 illustrates the hooks (the hooks are designated as the scattered dots and grey shaded portions) located on that side of the second connective left 18 and right 16 side strips. FIG. 3 also details the pull-tabs of the present invention which are located toward the top of the first connective side strips. The dashed line is representative of the boundary of the first connective side strip.

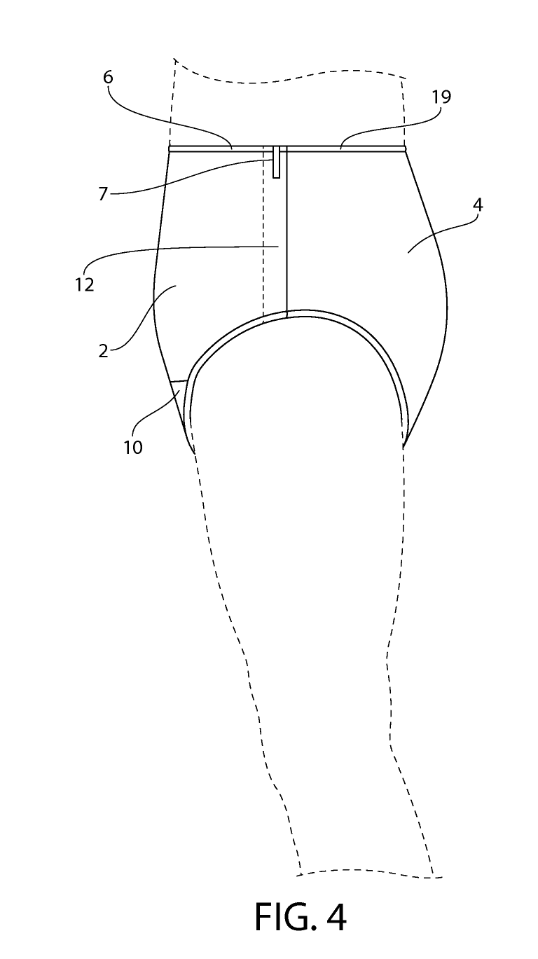

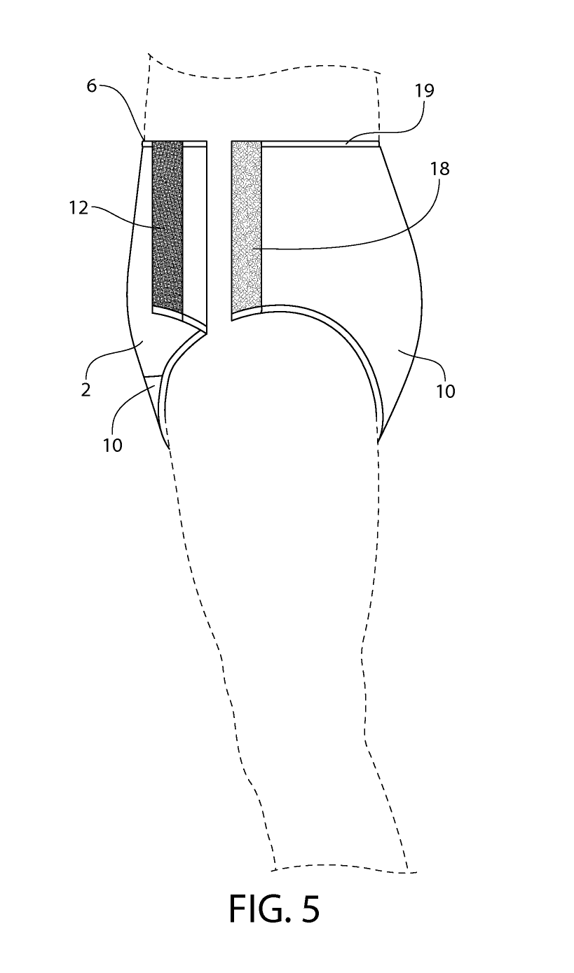

FIGS. 4 and 5 are side profile views of the women's underwear and are depicted as being worn by a user. FIGS. 4 and 5 illustrate how the connective side strips are attached to one another when worn by a user. In FIGS. 4 and 5 the front waistband 6 and back waistband 19 curve around the waist/hip area of the user. In FIG. 4 the first connective left side strip 12 is in a closed positioned. The left pull tab 7 allows a user to easily connect the connective side strips to one another. The dashed line inside the undergarment represents the boundary of the connective side strip. FIG. 5 illustrates the first connective left side strip 12 in an open position. The second connective left side strip 18 is positioned directly against the body of the user. The garment is securely fastened when both the first and second connective side strips are joined together as depicted in FIG. 4.

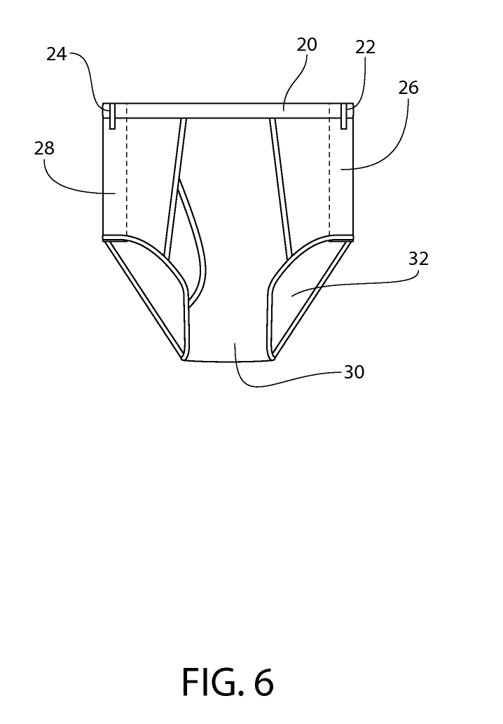

FIGS. 6-10 depict one embodiment of the present invention wherein the undergarment is a men's brief. The underwear can be made of woven, nonwoven or knitted fabric or any fabric or material that is normally used in traditional undergarments.

FIG. 6 is a front view of the men's briefs. It is one advantage of the present invention that the men's briefs contain left 22 and right 24 pull-tabs. The front waist band portion 20 is located toward the what is the waist area of a user and the first connective left side strip 26 and first connective right side strip 28 both allow the garment to be fastened and secure. The front 30 and rear 32 panels may be attached to one another by stitching or any other means known in the art. The dashed line is merely representative of the boundary of the connective side strips.

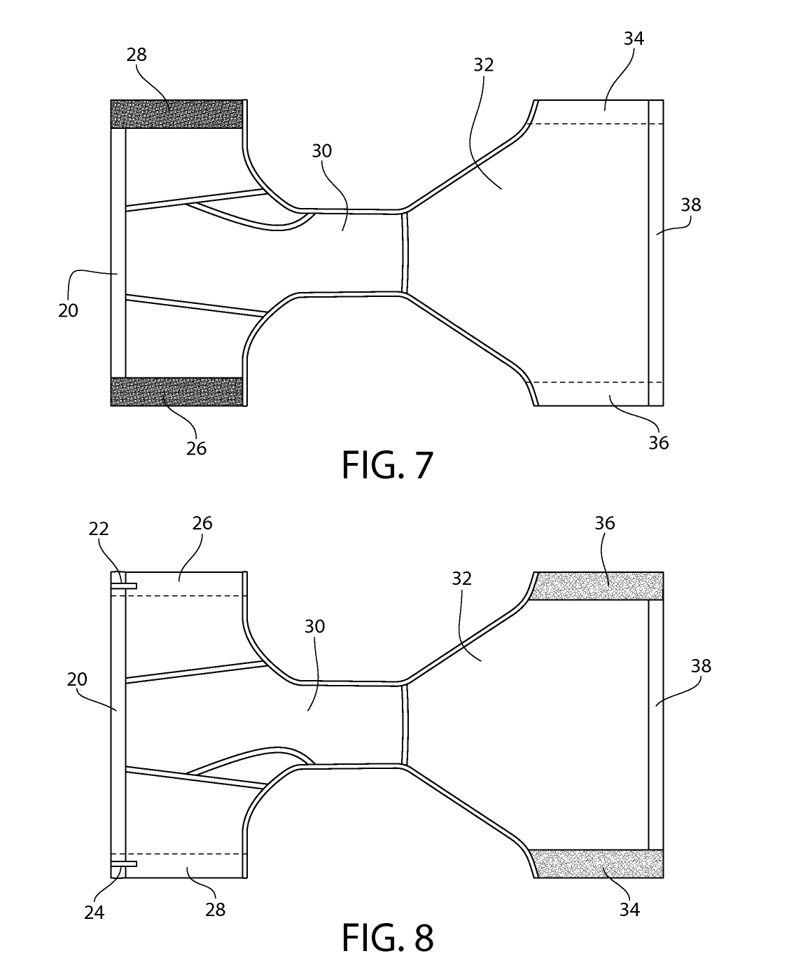

FIG. 7 is an above view of the interior of the men's brief. FIG. 8 is an above view of the exterior of the men's brief. FIGS. 7 and 8 depict the men's brief laid flat. In FIG. 7, the first connective left side strip 26 and first connective right side strip 28 allow the user to connect and fasten the undergarment together with second connective left 36 and right 34 side strips. The first connective left side strip 26 and first connective right side strip 28 both comprise connective means (loops which are designated by black shading). The second connective left side strip 36 and second connective right side strip 34 both comprise the corresponding hooks (not shown). The front 30 and rear 32 panels are still joined even when the connective strips are disengaged.

FIG. 8 is an above exterior view of the men's brief of the present invention. The figure illustrates the front 20 waistband portion as well as the back 38 waistband portion. FIG. 8 illustrates the second connective left side strip 36 as well as the second connective right side strip 34. Wherein the second connective left strip 36 and second connective right 34 side strip both comprise connective means which are hooks (the hooks are designated as the scattered dots or grey shading) and which correspond to the loops of the first connective left 26 and right 28 side strips. The dashed line in both FIG. 7 and FIG. 8 merely represents the boundaries of the respective connective side strips.

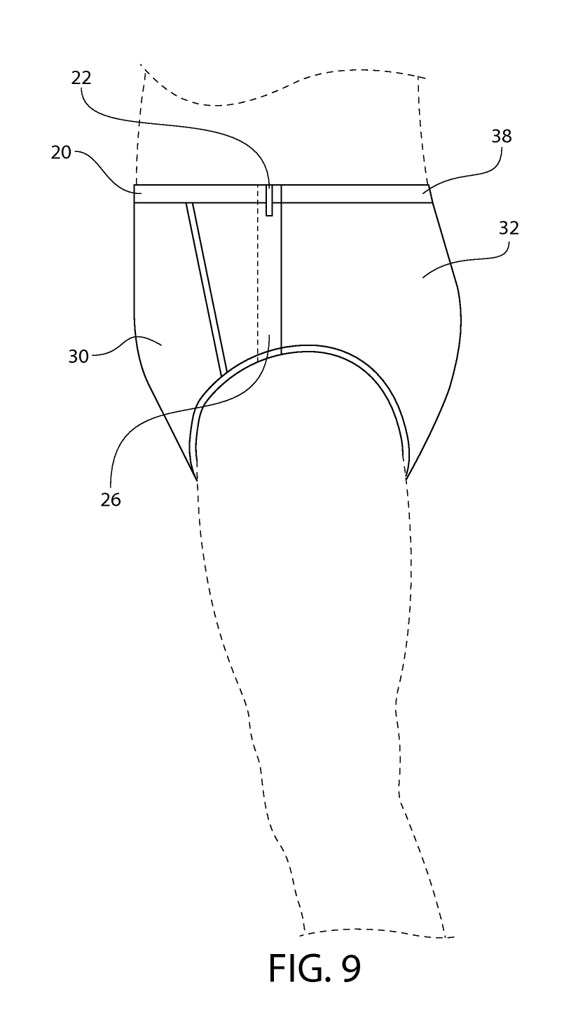

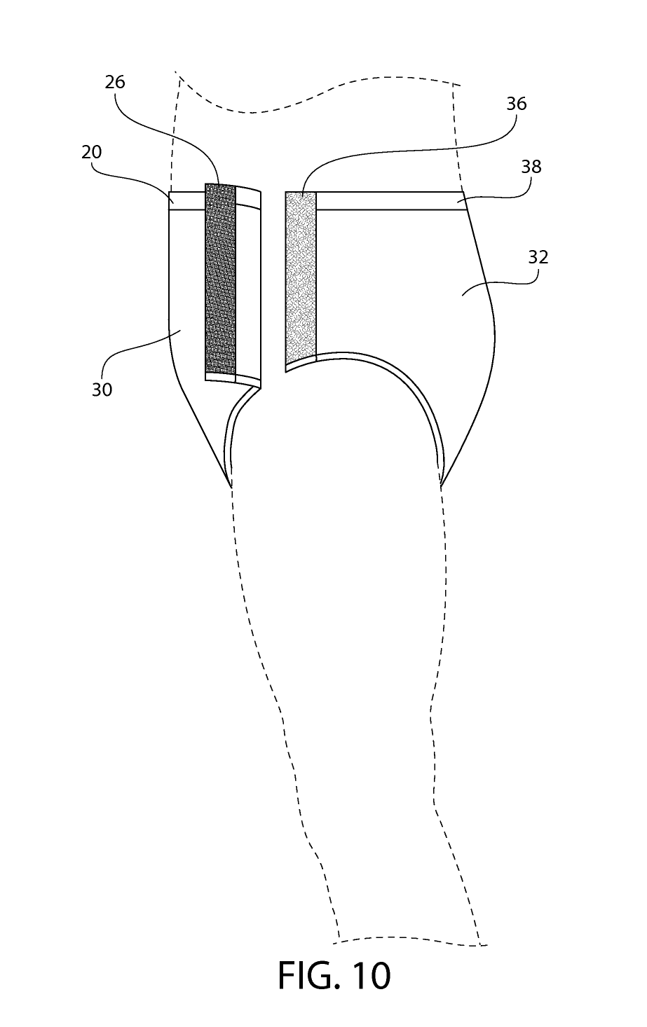

FIGS. 9 and 10 are side views of the men's briefs depicted as being worn by a user. FIGS. 9 and 10 illustrate how the connective side strips may be attached and disengaged to and from one another. In FIGS. 9 and 10 the front waistband portion 20 and back waistband portion 38 curve around the waist/hip area of the user. In FIG. 9 the first connective left side strip 26 is in a closed positioned and fastened to its complementary connective side strip (not shown). The left pull-tab 22 allows a user to easily connect the connective side strips to one another. FIG. 10 illustrates the first connective left side strip 26 in an open position. The second connective left side strip 36 is positioned against the body of the user. The undergarment is securely fastened when both the first and second connective side strips are joined together as depicted in FIG. 9. The dashed line of FIG. 9 is merely to illustrate the boundary of the connective side strip.

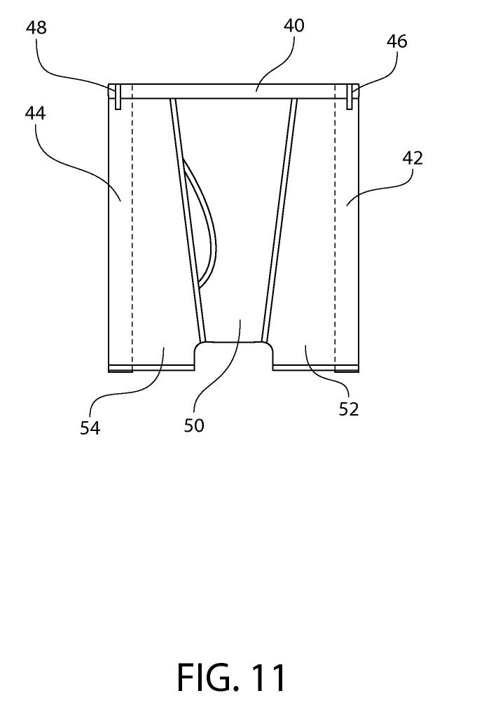

FIGS. 11-15 depict one embodiment of the present invention wherein the undergarment is a men's boxer brief. The underwear can be made of woven, nonwoven or knitted fabric or any fabric or material that is normally used in traditional undergarments.

FIG. 11 is a front view of the men's boxer brief. The boxer brief has a similar fit to traditional undergarment and has a front elastic band portion 40, and contains a front panel 50 which is attached to a back panel (not shown), front left panel 52, and front right panel 54, by stitching or any means of attachment known in the art. The figure depicts a waistband 40 in addition to a first connective left side strip 42 and a first connective right side strip 44. The figure details the left pull tab 46, and the right pull tab 48, of the men's boxer brief. The left 46 and right 48 pull tabs, allow for an individual to easily remove the undergarment and with limited movement. The front middle 50, front left 52, and front right 54 strips are connected to one another by stitching or other means known in the art. The dashed line is meant to merely illustrate or represent the boundary of the connective strips.

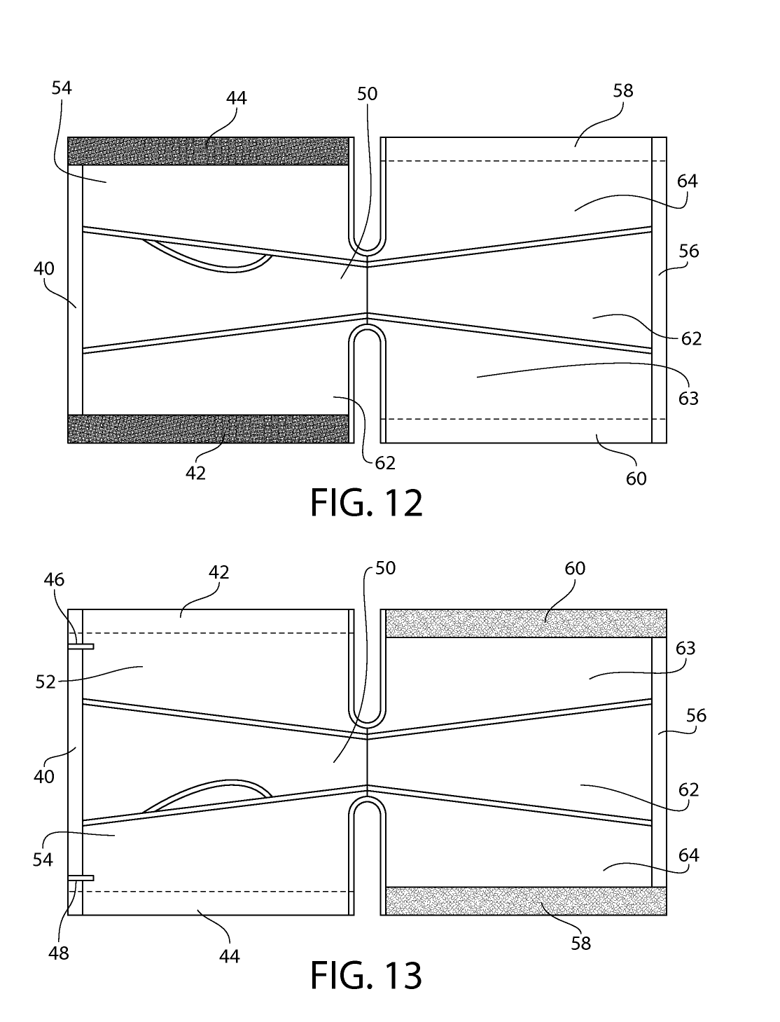

FIG. 12 is an above view of the interior of the men's boxer brief. FIG. 13 is an above view of the exterior of the men's boxer brief. FIGS. 12 and 13 depict the men's brief laid flat. In FIG. 12, the first 42 connective left side strip and first 44 connective right side strip of the undergarment allow the user to connect the undergarment together at around the waist or hip area with the complementary second connective left 60 and second connective right 58 side strips. FIG. 12 displays the loops (the loops are designated by black shading) of the garment. The second connective left 60 side strip and second connective right side strip 58 both comprise the connective means (not shown in FIG. 12) comprised by the second connective left side strip 60 and second connective right side strip 58.

FIG. 13 is an above view of the exterior of the men's boxer brief. The figure illustrates the front 40 waistband portion as well as the back 56 waistband portion. FIG. 13 illustrates the second connective left side strip 60 as well as the second connective right side strip 60. Wherein the left 60 and right 58 second connective side strips both comprise connective means which are hooks (the hooks are designated as the scattered dots or grey shading) and which correspond to the connective means (not shown in FIG. 13) of the first connective left 42 and right 44 side strips. The back middle 62, left 63, and right 64 panels are connected to one another by stitching or any other means known in the art.

The dashed lines in FIG. 12 and FIG. 13 are merely representative of the boundaries of the respective connective side strips.

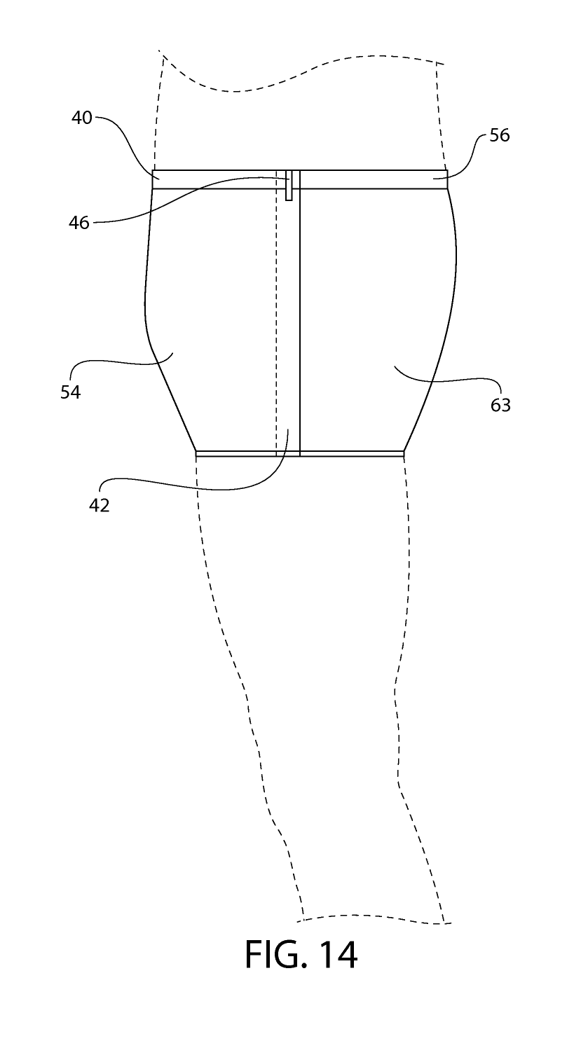

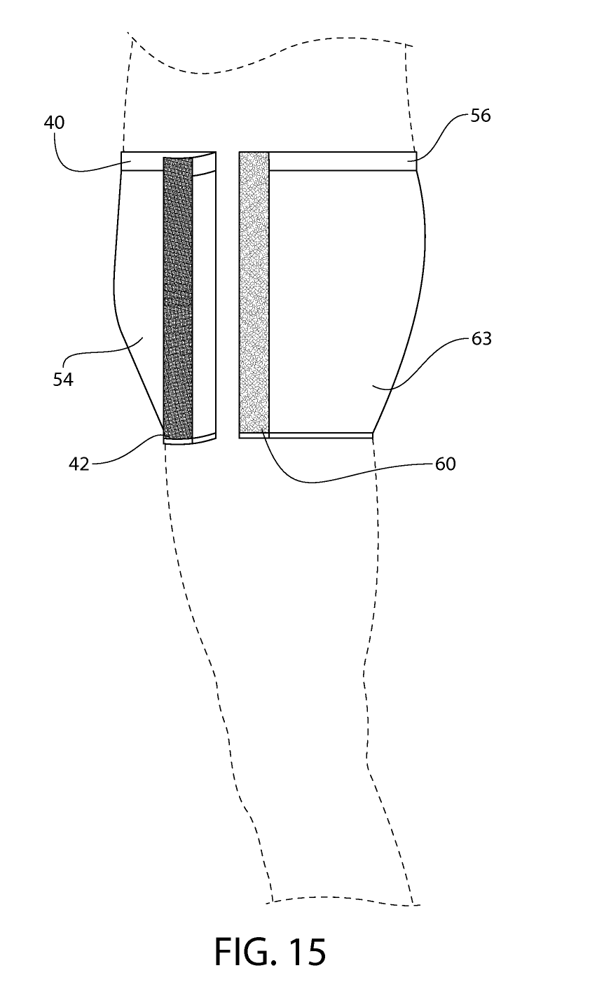

FIGS. 14 and 15 are side views of the men's boxer brief depicted being worn by a user. FIGS. 14 and 15 illustrate how the connective side strips are attached to one another. In FIGS. 14 and 15 the front waistband 40 portion and back waistband 56 portion curve around the waist/hip area of the user. In FIG. 14 the first connective left side strip 42 is in a closed positioned. The left pull-tab 46 allows a user to easily connect the or disconnect the complementary connective side strips to one another. FIG. 15 illustrates the first connective left side strip 42 in an open position and disengaged from the second connective left side strip 60. The second connective left side strip 60 is positioned against the body of the user. The garment is securely fastened when both the first 42 and second 60 connective side strips are joined together as displayed in the FIG. 14. In this embodiment the undergarment fits similarly to comparable undergarments that have not been modified to accommodate a user of limited mobility.

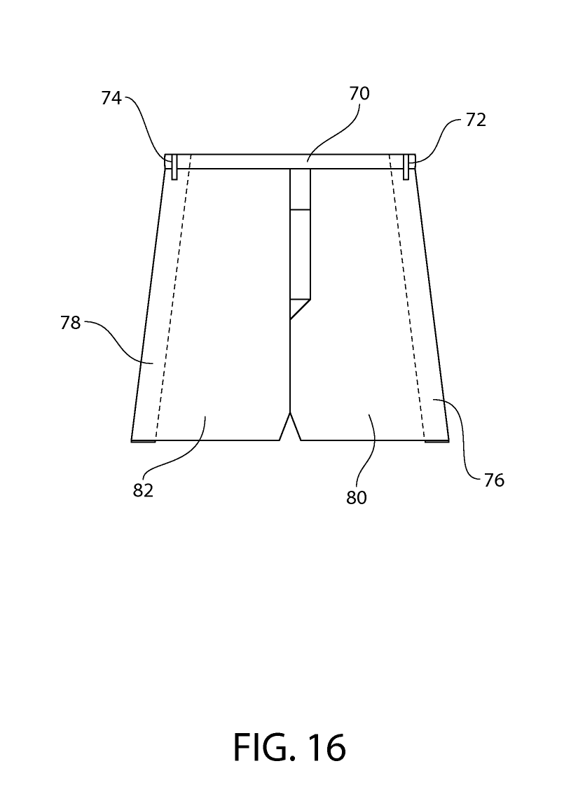

FIGS. 16-20 depict one embodiment of the present invention wherein the undergarment is men's boxer underwear. The underwear can be made of woven, nonwoven or knitted fabric or any fabric or material that is normally used in traditional undergarments.

FIG. 16 is front view of the men's boxer underwear. The underwear contains a front waist band 70 portion, and contains left 80 and right 82 front panels which are also attached to a back strip (not shown) by means of attachment known in the art (e.g., stitching). The figure depicts a first connective left side strip 76 and a first connective right 78 side strip. The figure details the left pull tab 72, and the right pull tab 74, of the men's boxer brief which are attached to the connective side panels. The left 72 and right 74 pull-tabs, allow for an individual to easily remove the underwear without needing very much mobility. The front left 80, and front right 82 strips are connected to one another by stitching or other means known in the art.

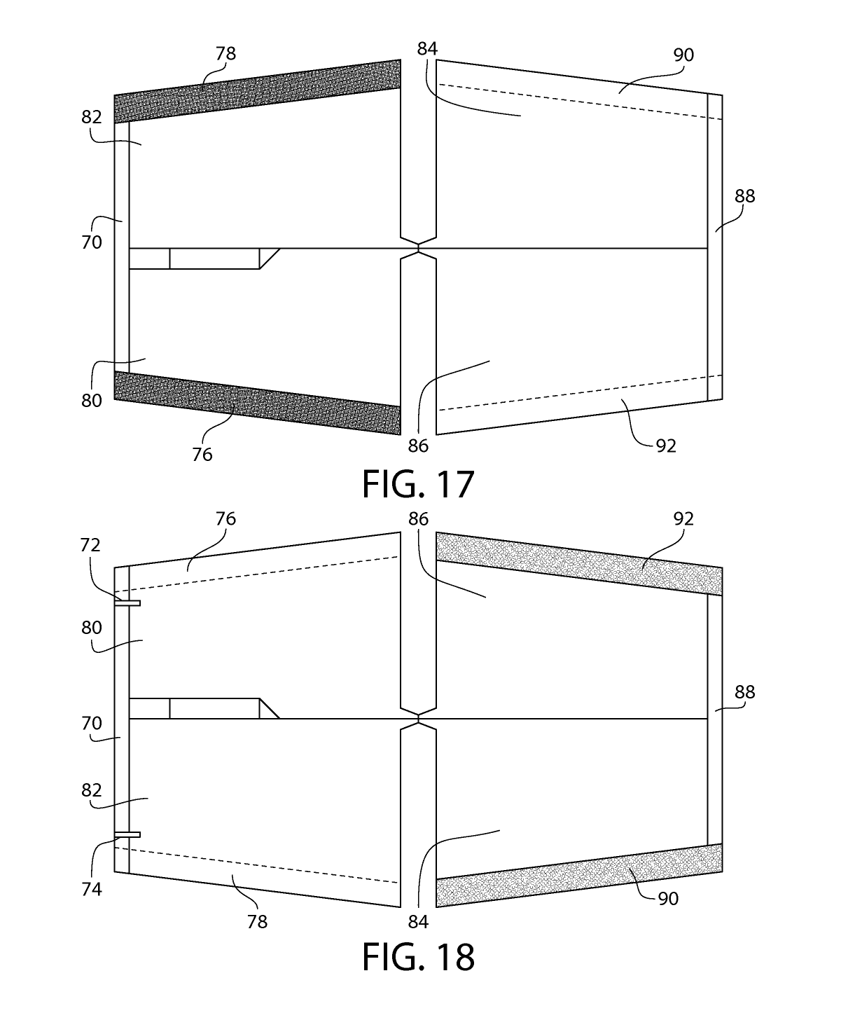

FIG. 17 is an above view of the interior of the men's boxer underwear. FIG. 18 is an above view of the exterior of the men's boxer underwear. FIGS. 17 and 18 depict the men's boxer underwear laid flat. In FIG. 17, the first connective left side strip 76 and first connective right side strip 78 of the garment allow the user to connect with the complementary second connective left side strip 92 and second connective right side strip 90. FIG. 17 illustrates that the first connective left side strip and first connective right side strip 78 each comprise the connective means which in this embodiment are loops (the loops are designated by black shading). The second connective left 92 and right 90 side strips each comprise the corresponding connective means, e.g., hooks (not shown in FIG. 17).

FIG. 18 is an above view of the exterior of the men's boxer underwear. The figure illustrates the front 70 waistband portion as well as the back 88 waistband portion. FIG. 18 illustrates the second connective left side strip 92 as well as the second connective right side strip 90. Wherein both the second connective left 92 and right 90 side strips comprise connective means, which in this embodiment are hooks (the hooks are designated as the scattered dots or grey shading). The hooks correspond to the loops (designated by black shading in FIG. 17) comprised by the first connective left 76 and right 78 side strips. The back left 86, and back right 84 panels are connected to one another by stitching or any other means known in the art. As depicted, the undergarment remains as one piece even when the connective side strips are not engaged or fastened.

The dashed lines in FIG. 17 and FIG. 18 are merely representative of the boundary of the respective connective side strips.

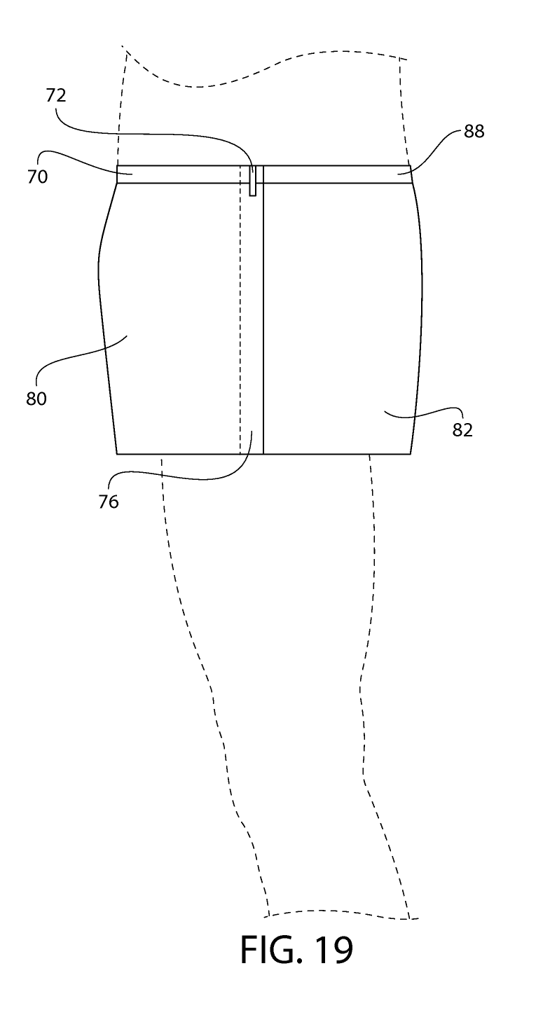

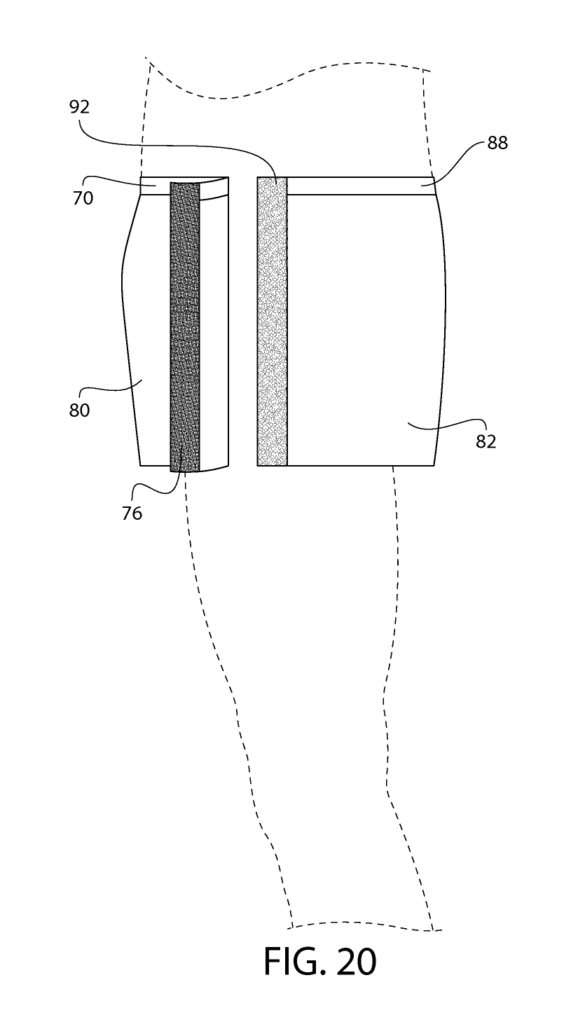

FIGS. 19 and 20 are side views of the men's boxer underwear depicted as being worn by a user. FIGS. 19 and 20 illustrate how the connective side strips are attached to one another. In FIGS. 19 and 20 the front waistband 70 portion and back waistband 88 portion curve around the waist/hip area of the user. In FIG. 19 the first connective left side strip 76 is in a closed positioned and is shown engaged with the second connective left side strip (not shown). The left pull tab 72 allows a user to easily connect (or disconnect) the complementary first and second connective side strips to one another by inserting a thumb or finger through the pull tab loop and applying a force necessary to bring the connective strips together or pull them apart. FIG. 20 illustrates the first connective left side strip 76 in an open position and disengaged from the corresponding second connective left side strip 92. The second connective left side strip 92 is positioned against the body of the user. The garment is securely fastened when both the first and second connective side strips are joined together as displayed in FIG. 19.

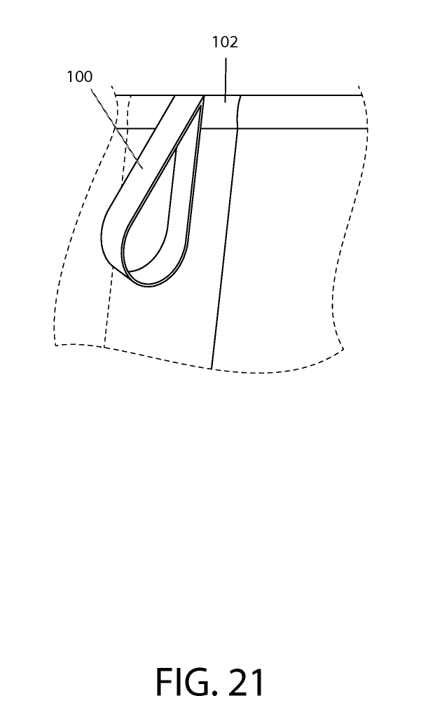

FIG. 21 is an exploded or detailed view of the top portion of a pull-tab system. Similar pull-tab systems are disclosed in FIGS. 1-20. The pull-tab element is depicted as attached to the top portion of a connective strip, in this embodiment where the waistband and the connective strip overlap. FIG. 21 illustrates that the tab may placed on the medial portion of the connective strip which in certain embodiments can allow for the greatest control and manipulation of the connective side strip and allows a person with limited mobility to have an easier grip and more control when manipulating the connective panel. The placement of the tab loop also allows an individual to control put on or remove the article of clothing (e.g., garment or undergarment) using only minimal or limited physical movement.

Detailed embodiments of the present invention are disclosed herein; however, it is to be understood that the disclosed embodiments, examples, figures are merely illustrative of the invention that may be embodied in various forms. In addition, each of the examples given in connective with the various embodiments of the invention are intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

1. A removable undergarment that may be easily removed or worn by an individual with limited mobility wherein the removable undergarment comprises:
a.) a natural or synthetic material or fabric,
b.) one or more connective strips, and wherein the connective strips further comprise:
i.) connective means and
ii.) a pull tab;
Wherein at least one pull tab is attached to at least one connective strip, and
wherein the pull tab is optionally attached to the medial portion of the connective strip and wherein an applied force on the pull tab is sufficient to fasten or disengage a first connective side strip from a second complementary connective strip.

2. The removable undergarment of claim 1, wherein said connective means are hook and loop fasteners.

3. The removable undergarment of claim 1, wherein said connective strips include:
a.) a first connective left strip;
b.) a first connective right strip;
c.) a second connective left strip; and
d.) a second connective right strip;
wherein the first connective left strip and second connective left strips are complementary with one another; and wherein the second connective left strip and second connective right strips are complementary with one another.

4. A removable garment that is easily removed or worn by an individual with limited mobility, wherein the garment comprises:
a.) a natural or synthetic material or fabric and;
b.) one or more connective strips, wherein the connective strips further comprise:
i.) connective means
and;
ii.) a pull tab;
wherein at least one pull tab is attached to at least one connective strip, and
wherein the pull tab is optionally attached to the medial of the connective strip and wherein an applied force on the pull tab is sufficient to fasten or disengage a connective side strip from a second complementary connective strip.

5. The removable garment of claim 4, wherein said connective means are hook and loop fasteners.

6. The removable garment of claim 4, wherein said connective strips include:
a.) a first connective left strip;
b.) a first connective right strip;
c.) a second connective left strip; and
d.) a second connective right strip;
wherein the first connective left strip and second connective left strips are complementary with one another; and wherein the second connective left strip and second connective right strips are complementary with one another.

7. A pull-tab System that may be combined with an article of clothing or accessory in order to make the clothing or accessory easier to wear for an individual with limited mobility, wherein the system comprises:
a.) one or more pull-tabs and;
b.) one or more connective strips pairs, and wherein the connective strips further comprise
i.) corresponding or complementary connective means.
wherein at least one pull tab is attached to at least one connective strip, and wherein an applied force on the pull tab is sufficient to fasten or disengage a first connective side strip from its complementary second connective side strip.

8. The undergarment of claim 1, wherein the undergarment comprises a pad.

9. The garment of claim 4, wherein the garment comprises a pad.
